(19) United States
(12) Patent Application Publication
(10) Pub. No.: US 2012/0071742 A1
(43) Pub. Date: Mar. 22, 2012

(54) REMANUFACTURING A STACKED ADHESIVE MEDICAL SENSOR

(75) Inventors: Casey V. Medina, Westminster, CO (US); David P. Besko, Thornton, CO (US); Charles Haisley, Boulder, CO (US)

(73) Assignee: Nellcor Puritan Bennett LLC, Boulder, CO (US)

(21) Appl. No.: 12/888,248

(22) Filed: Sep. 22, 2010

(51) Int. Cl. A61B 5/1455; B23P 6/00

(52) U.S. Cl. 600/344; 29/402.01

(57) ABSTRACT

Remanufactured medical sensors and methods for remanufacturing used stacked adhesive medical sensors are provided. Such a remanufactured sensor may include certain components from a used stacked adhesive medical sensor and certain new components. For example, a remanufactured medical sensor may include an exterior foam layer, a mask layer, an emitter and a detector, a semi-rigid optical mount to hold the emitter and the detector in place, optical windows, and an interior foam layer. At least the emitter and the detector may derive from the used stacked adhesive medical sensor, while at least one of the exterior foam layer, the mask layer, the semi-rigid optical mount, the optical windows, or the interior foam layer may be new.

### BACKGROUND

The present disclosure relates generally to remanufacturing disposable medical sensors and, more particularly, to remanufacturing, i.e., reconstructing, used stacked adhesive medical sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of devices have been developed for non-invasively monitoring physiological characteristics of patients. For example, a pulse oximetry sensor system may non-invasively detect various patient blood fluid characteristics, such as the blood-oxygen saturation of hemoglobin in arterial blood, the volume of individual blood pulsations supply the tissue, and/or the rate of blood pulsations corresponding to each heart beat of a patient. To determine these physiological characteristics, light may be emitted into patient tissue, where the light may be scattered and/or absorbed in a manner dependent on such physiological characteristics.

Many pulse oximeter medical sensors may be disposable and originally intended for use on a single patient. One such disposable medical sensor may be a stacked adhesive medical sensor such as the Max-Fast sensor by Nellcor, which may include multiple stacked adhesive layers for multiple reapplications onto patient tissue. In particular, a medical practitioner may first attach a stacked adhesive medical sensor to a patient tissue site via an outermost of the stacked adhesive layers. When the medical practitioner checks the sensor site at a later time, causing the medical sensor to lift away from the patient tissue, the outermost adhesive layer may be removed to expose a new, fresh adhesive layer underneath. The stacked adhesive medical sensor may be reattached to the tissue site using the newly exposed adhesive layer. When all of the adhesive layers have been removed or when the patient no longer needs monitoring, the stacked adhesive medical sensor is discarded.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

Embodiments of the present disclosure relate to remanufactured medical sensors and methods for remanufacturing used stacked adhesive medical sensors. Such a remanufactured sensor may include certain components from a used stacked adhesive medical sensor and certain new components. For example, a remanufactured medical sensor may include an exterior foam layer, a mask layer, an emitter and a detector, a semi-rigid optical mount to hold the emitter and the detector in place, optical windows, and an interior foam layer. At least the emitter and the detector may derive from the used stacked adhesive medical sensor, while at least one of the exterior foam layer, the mask layer, the semi-rigid optical mount, the optical windows, or the interior foam layer may be new.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. Again, the brief summary presented above is intended only to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings in which:

FIG. 1 is a perspective view of a medical sensor system having a remanufactured stacked adhesive medical sensor, in accordance with an embodiment;

FIG. 2 is a more detailed view of the remanufactured stacked adhesive medical sensor of FIG. 1, in accordance with an embodiment;

FIG. 3 is a perspective view of a headband for securing the remanufactured stacked adhesive medical sensor of FIG. 2, in accordance with an embodiment;

FIG. 4 is an exploded view of a remanufactured stacked adhesive sensor having one or more new adhesive layers attached to existing components of a used stacked adhesive medical sensor, in accordance with an embodiment;

FIG. 5 is an exploded view of a remanufactured stacked adhesive medical sensor having a cover layer and one or more stacked adhesive layers attached to existing components of a used stacked adhesive medical sensor, in accordance with an embodiment;

FIGS. 6-7 are remanufactured stacked adhesive medical sensors having a cover layer and one or more stacked adhesive layers attached to existing components of a used stacked adhesive medical sensor, in accordance with embodiments;

FIG. 7 is an exploded view of a remanufactured stacked adhesive medical sensor having one or more stacked adhesive layers and an outer covering layer attached to existing components of a used stacked adhesive medical sensor, in accordance with an embodiment;

FIG. 8 is a flowchart describing an embodiment of a method for remanufacturing a used stacked adhesive medical sensor to obtain a remanufactured stacked adhesive medical sensor of FIGS. 4-7;

FIG. 9 is an exploded view of a remanufactured stacked adhesive medical sensor having a non-adhesive covering layer attached to existing components of a used stacked adhesive medical sensor, in accordance with an embodiment;

FIG. 10 is an exploded view of a remanufactured stacked adhesive medical sensor having a non-adhesive covering layer covered by adhesive dots that is attached to existing components of a used stacked adhesive medical sensor, in accordance with an embodiment;

FIG. 11 is an exploded view of a remanufactured stacked adhesive medical sensor having a cover layer and an outer headband-coupling layer attached to existing components of a used stacked adhesive medical sensor, in accordance with an embodiment;

FIG. 12 is a flowchart describing an embodiment of a method for remanufacturing a used stacked adhesive medical sensor to obtain a remanufactured stacked adhesive medical sensor of FIGS. 9-11;

FIG. 13 is an exploded view of a remanufactured stacked adhesive medical sensor having a new foam layer and one or more stacked adhesive layers attached to existing components of a used stacked adhesive medical sensor, in accordance with an embodiment;

FIG. 14 is a flowchart describing an embodiment of a method for remanufacturing a used stacked adhesive medical sensor to obtain a remanufactured stacked adhesive medical sensor of FIG. 13;

FIG. 15 is an exploded view of a remanufactured stacked adhesive medical sensor having a new foam layer, one or more stacked adhesive layers, outer layer, and mask layer attached to existing components of a used stacked adhesive medical sensor, in accordance with an embodiment;

FIG. 16 is a flowchart describing an embodiment of a method for remanufacturing a used stacked adhesive medical sensor to obtain a remanufactured stacked adhesive medical sensor of FIG. 15;

FIG. 17 is an exploded view of a remanufactured stacked adhesive medical sensor in which only certain electronic components such as an emitter and detector from a used stacked adhesive medical sensor are attached to other new components, in accordance with an embodiment; and

FIG. 18 is a flowchart describing an embodiment of a method for remanufacturing a used stacked adhesive medical sensor to obtain a remanufactured stacked adhesive medical sensor of FIG. 17.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Also, as used herein, the term “over” or “above” refers to a component location on a medical sensor that is closer to patient tissue when the medical sensor is applied to the patient. For example, a stacked adhesive layer of a stacked adhesive medical sensor may be understood to be “over” or “above” the emitter or detector, as will be described below.

Present embodiments relate to remanufacturing used stacked adhesive medical sensors. As discussed above, a stacked adhesive medical sensor, when new, may include several stacked layers of adhesive that may be individually removed to extend the life of the medical sensor. By way of example, the Max-Fast pulse oximeter sensor by Nellcor represents such a stacked adhesive medical sensor. These medical sensors are generally known to be one-time-use medical sensors that may be disposed after use by one patient or after all of the stacked adhesive layers have been used. Though disposable, some components of used stacked adhesive medical sensors may be employed in the reconstruction of stacked adhesive medical sensors. As discussed in greater detail below, such components may include, for example, a cable, memory, an emitter and detector, components that hold the emitter and detector in place, and various layers that surround the emitter and detector. Reusing such components to reconstruct a stacked adhesive medical sensor may reduce waste, consequently reducing an impact on the environment, while accordingly reducing costs.

Thus, embodiments of the present disclosure involve remanufacturing used stacked adhesive medical sensors that may have various reusable components. For example, in some embodiments, foam layers encapsulating an emitter and detector may be reused while new stacked adhesive layers are replaced, with or without a new non-patient-contacting-side covering layer. In other embodiments, no stacked adhesive layers may be added, but other adhesives, such as gels, bandages, and/or adhesive dots may be applied to the covering of patient-contacting-side covering layer or headband-adhering-material may be attached to the exterior of a used stacked adhesive sensor. In certain embodiments, the electronic components and a non-patient-contacting side foam layer may be reused from a used stacked adhesive medical sensor and a patient-contacting-side foam layer and the stacked adhesive layers may be replaced. In still other embodiments, only certain electronic components such an emitter and a detector and/or accompanying components may be reused and all other layers may be replaced. The embodiments discussed above also may reuse certain electrical components, such as a cable, connector, and memory, which may be used to join the emitter and the detector to a patient monitor.

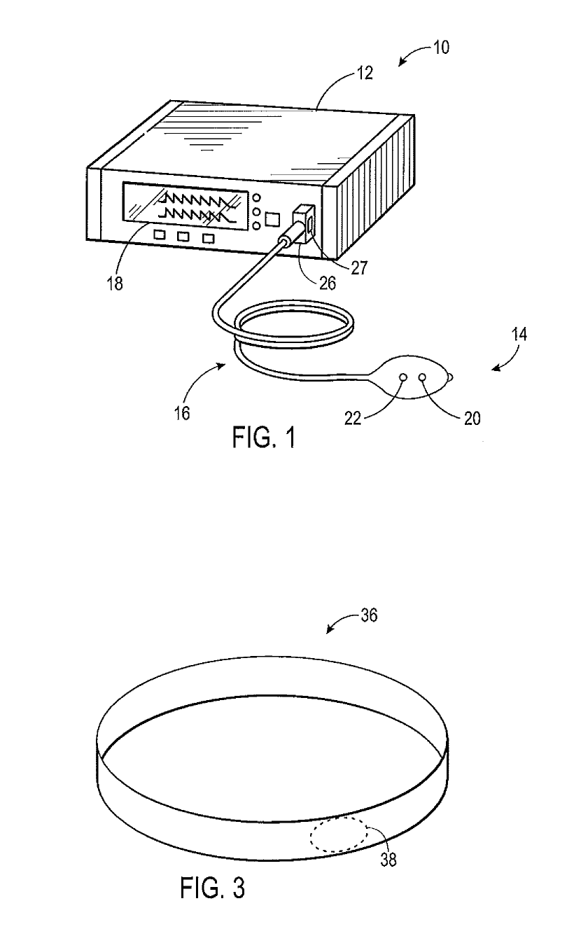

With the foregoing in mind, FIG. 1 illustrates a perspective view of an embodiment of a non-invasive medical sensor system 10 involving an electronic patient monitor 12 and a remanufactured stacked adhesive medical sensor 14. By way of example, the patient monitor 12 may be a patient monitor by Nellcor or another manufacturer. The remanufactured stacked adhesive medical sensor 14 may be remanufactured, as discussed below, from a stacked adhesive medical sensor that has been used and/or discarded. The patient monitor 12 may exchange signals with the remanufactured stacked adhesive medical sensor 14 via a communication cable 16. The patient monitor 12 may include a display 18, a memory 27 (which may be located in a connector 26 of the cable 16), and various monitoring and control features. In certain embodiments, the patient monitor 12 may include a processor that may determine a physiological parameter of a patient based on these signals obtained from the remanufactured stacked adhesive medical sensor 14. Indeed, in the presently illustrated embodiment of the system 10, the remanufactured stacked adhesive medical sensor 14 is a pulse oximetry sensor that may non-invasively obtain pulse oximetry data from a patient.

The stacked adhesive medical sensor 14 may attach to pulsatile patient tissue (e.g., a patient's forehead). An emitter 20 and a detector 22 may operate to generate non-invasive pulse oximetry data for use by the patient monitor 12. In particular, the emitter 20 may transmit light at certain wavelengths into the tissue and the detector 22 may receive the light after it has passed through or is reflected by the tissue. The amount of light and/or certain characteristics of light waves passing through or reflected by the tissue may vary in accordance with changing amounts of blood contingents in the tissue, as well as related light absorption and/or scattering.

The emitter 20 may emit light from two or more light emitting diodes (LEDs) or other suitable light sources into the pulsatile tissue. The light that is reflected or transmitted through the tissue may be detected using the detector 22, which may be a photodetector (e.g., a photodiode), once the light has passed through or has been reflected by the pulsatile tissue. When the detector 22 detects this light, the detector 22 may generate a photocurrent proportional to the amount of detected light, which may be transmitted through the cable 16 to the patient monitor 12. The patient monitor 12 may convert the photocurrent from the detector 22 into a voltage signal that may be analyzed to determine certain physiological characteristics of the patient.

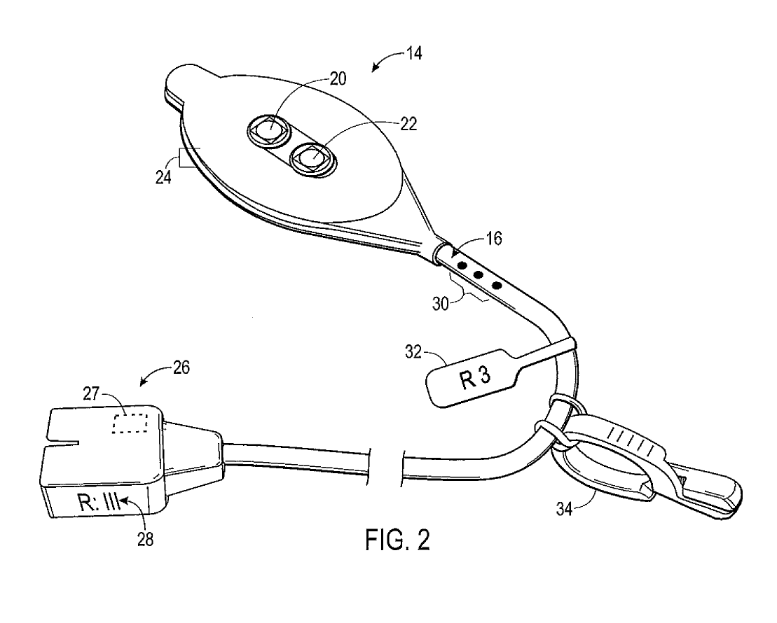

The remanufactured stacked adhesive medical sensor 14 may include certain new components and certain existing components from a used stacked adhesive medical sensor. Turning to FIG. 2, for example, the emitter 20 and detector 22, as well as one or more layers 24 of the remanufactured stacked adhesive medical sensor 14 may derive from a used stacked adhesive medical sensor. A connector 26 attached to the medical cable 16 may include memory 27 with patient trend data that has been erased or disabled. Additionally, the memory 27 stored in the connector 26 may include a recycle indicator to note how many times the remanufactured stacked adhesive medical sensor 14 has been remanufactured.

Additionally or alternatively, other recycle indicators may be present on the remanufactured stacked adhesive medical sensor 14. For example, embossing 28 may represent one recycle indicator, here indicating three remanufacturing times. Ink marks 30 on the cable 16, on the layers 24, or any other location may provide a similar indication, as may a cord tag 32. A cord clip 34 may be new or used and may attach to patient clothing or bedding during operation to secure this remanufactured stacked adhesive medical sensor 14 to the patient. In some embodiments, the clip 34 and/or an outermost non-patient-contacting-side layer 24 may indicate how many times the remanufactured stacked adhesive medical sensor 14 has been remanufactured (e.g., via ink marks or a specific coloring).

In certain embodiments, the remanufactured stacked adhesive medical sensor 14 may be held in place on a patient tissue site by an elastic headband 36, as shown in FIG. 3. The elastic headband 36 may be new or may be remanufactured through sterilization and/or laundering, and may or may not include a specific sensor-contacting site 38 to hold the remanufactured stacked adhesive medical sensor 14 in place on the patient's forehead. In addition, the elastic headband 36 may be included in a medical sensor “assembly” or package that includes at least one manner of attaching the remanufactured stacked adhesive medical sensor 14 to a patient tissue site. Such manners of attaching the remanufactured stacked adhesive medical sensor 14 to the patient tissue site should be understood to include those disclosed herein (e.g., adhesive gels, dots, bandages, and so forth), including by way of the elastic headband 36.

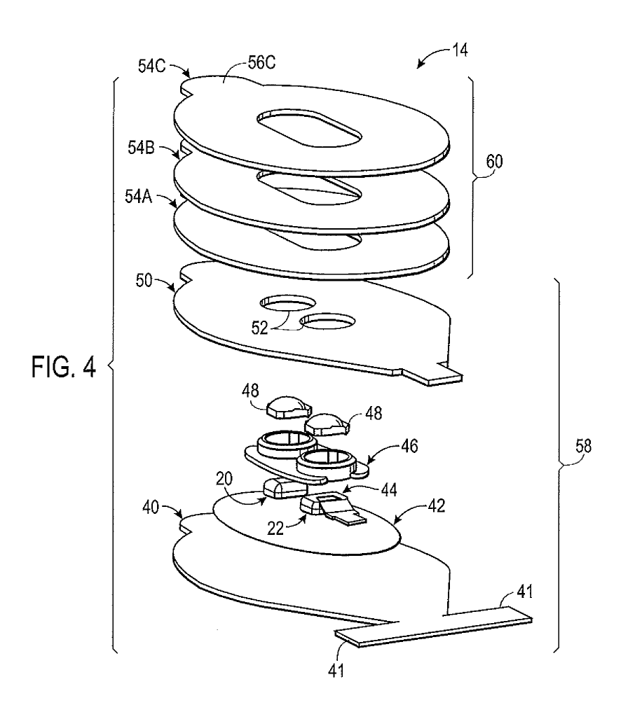

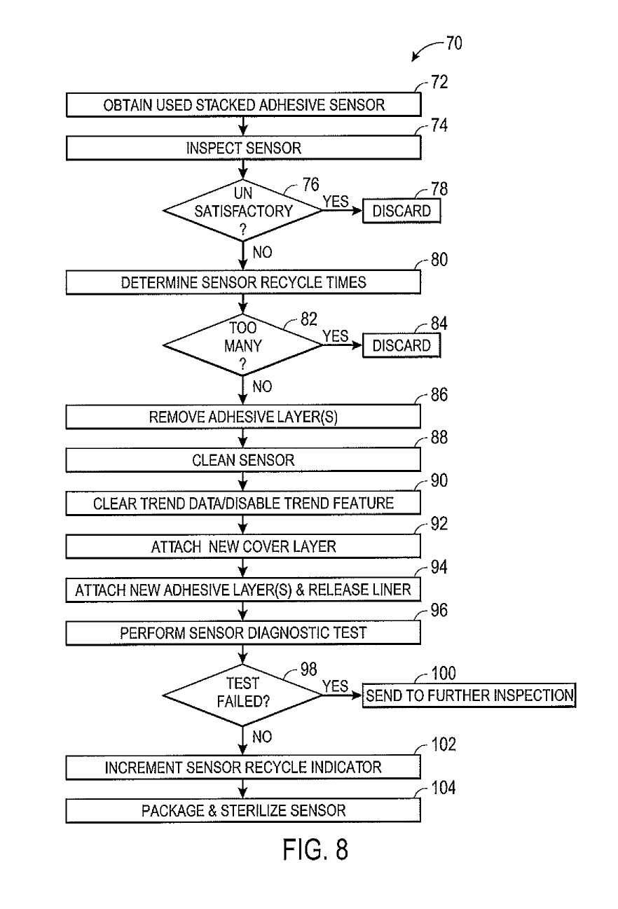

As mentioned above, the remanufactured stacked adhesive medical sensor 14 may reuse certain existing components from a stacked adhesive medical sensor that has been used and/or discarded. Indeed, all of the embodiments of the remanufactured stacked adhesive medical sensor 14 may reuse the communication cable 16, the associated connector 26, and memory 27. In particular, the embodiments represented in FIGS. 4-7 reuse at least the electronic components and two foam layers that surround the electronic components. One embodiment of a remanufacturing process for producing the remanufactured stacked adhesive medical sensors 14 shown in FIGS. 4-7 is discussed below with reference to FIG. 8

As shown in FIG. 4, one embodiment of the remanufactured stacked adhesive medical sensor 14 may include an outermost non-patient-contacting-side exterior foam layer 40 (which also may include a graphic logo layer to identify the remanufactured stacked adhesive medical sensor 14). The exterior foam layer 40 may have foam wings 41 to wrap around and attach to the cord 16. By way of example, the exterior foam layer 40 may be fabricated from a common PVC foam or a urethane foam material, such as the PORON™ family of urethanes commercially available from the Rogers Corporation of Connecticut. Attached to the exterior foam layer 40 may be a reflective mask layer 42 that may include, for example, an aluminized polypropylene film with a synthetic adhesive layer for attachment to the exterior foam layer 40. The mask layer 42 may prevent ambient light from passing through the exterior foam layer 40 and entering the detector 22.

Both the emitter 20 and the detector 22 may be attached to the mask layer 42. A Faraday shield 44 may be present around the detector 22 to reduce the effect of electrical fields on resulting photodetector signals. A semi-rigid optical mount 46 also may be present on the mask layer 42, surrounding and holding the emitter 20 and the detector 22 in a fixed manner while allowing a certain minimal amount of flexing and twisting to occur. This semi-rigid optical mount 46 also may be referred to as a “kayak” because of the way it holds the emitter 20 and the detector 22 in place. It should be noted that the semi-rigid optical mount 46 may prevent torque from causing orientation changes between the emitter 20 and the detector 22, which might interfere with the accuracy of measurements obtained by the detector 22 due to motion-induced artifacts and changes in calibration. Moreover, the optical mount 46 may serve as a shunt barrier between the emitter 20 and detector 22, and may be made of a black polypropylene material. Transparent windows 48 may couple to the semi-rigid optical mount 46 over the emitter 20 and detector 22.

A patient-contacting-side interior foam layer 50 may attach to the exterior foam layer 40 such that cutouts 52 fit around the optical windows 48 over the emitter 20 and detector 22. Like the exterior foam layer 40, the interior foam layer 50 may be formed from PVC foam or a urethane foam such as the as the PORON™ family of urethane foams. The interior foam layer 50 may attach to the exterior foam layer 40 using a pressure-sensitive adhesive or any other suitable adhesive. A number of stacked adhesive layers 54A-C may attach to the interior foam layer 50. The lowermost stacked adhesive layer 54A may attach to the interior foam layer 50 through an acrylic transfer adhesive or another suitable form of adhesive. Although only three stacked adhesive layers 54A-C are shown, it should be appreciated that any suitable number of stacked adhesive layers 54 may be present on the stacked adhesive medical sensor 14.

As shown in FIG. 4, the stacked adhesive layers 54A-C are each formed in a ring shape such that no adhesive is present between the emitter 20 and detector 22. This lack of intervening adhesive between the emitter 20 and detector 22 may reduce optical shunting, which may otherwise lead to measurement in accuracies. In some embodiments, the stacked adhesive layers 54A-C may be formed of a polyethylene film having an acrylic adhesive on one side for attachment to patient tissue. In certain other embodiments, the stacked adhesive layers 54A-C may have a thin layer of adhesive on both sides, in which case the adhesive layers may be separated from one another by release layers (not shown). Certain tab portions 56 of the stacked adhesive layers 54A-C may lack adhesive, which may allow a medical practitioner to easily remove the topmost of the stacked adhesive layers 54 (e.g., 54C) to expose a fresh stacked adhesive layer 54 below (e.g., 54B). In some embodiments, the lowest new stacked adhesive layer (e.g., 54A) may not include such a tab, indicating that the lowest new layer 54A should never be peeled back by a caregiver or patient. The stacked adhesive layers 54A-C may be colored so as to reduce the amount of ambient light that may enter the photodetector, (e.g., black). In addition, the stacked adhesive layers 54A-C may be constructed by polyester, polyimide, or Teflon, or any other suitable material, and the adhesive used on the surfaces of the stacked adhesive layers 54A-C may acrylic, synthetic rubber, natural rubber (e.g., latex), or any other suitable non-toxic adhesive. In other embodiments, the stacked adhesive layers 54A-C may not form a ring shape, but rather may include a continuous adhesive surface having a black strip between the emitter and detector to provide for optical shunting.

As noted above, the remanufactured stacked adhesive medical sensor 14 of FIG. 4 may include certain existing components (referred to by reference numeral 58) from a used stacked adhesive sensor and certain new components (referred to by reference numeral 60). In the embodiment of FIG. 4, the components between and including the exterior foam layer 40 and the interior foam layer 50 are existing components 58 from a used stacked adhesive medical sensor. However, at least one or more of the stacked adhesive layers 54A-C represents a new component 60 that has been added during the remanufacturing process.

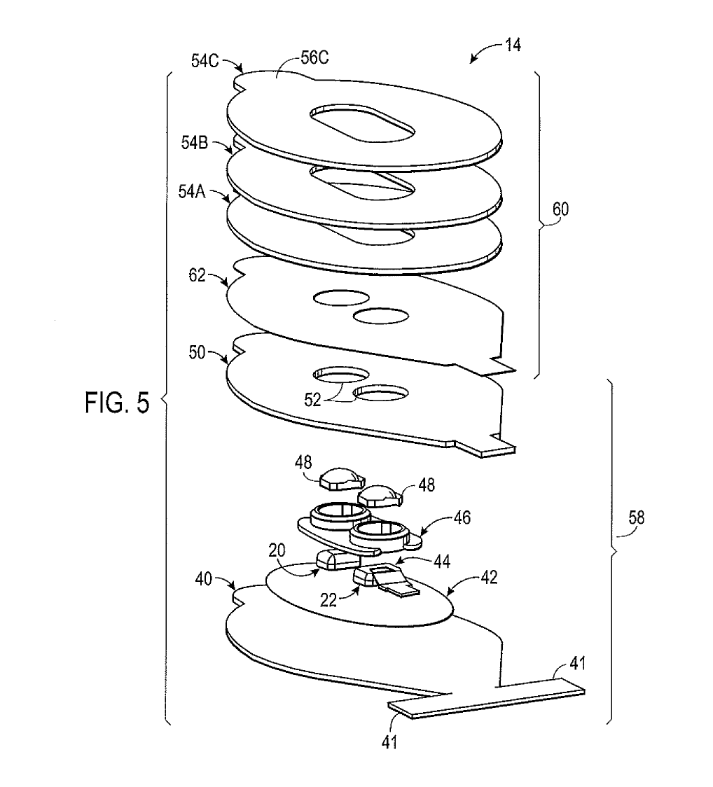

Similarly, FIG. 5 represents another embodiment of a remanufactured stacked adhesive medical sensor 14 having certain existing components 58 from a used stacked adhesive medical sensor and certain new components 60 added during the remanufacturing process. In the embodiment of FIG. 5, as in the embodiment of FIG. 4, all of the components between and including the exterior foam layer 40 and the interior foam layer 50 are existing components 58 from a used stacked adhesive medical sensor. Unlike the embodiment of FIG. 4, however, a patient-contacting-side interior cover layer 62 is formed over the interior foam layer 50. This new interior cover layer 62 may insure that no existing components 58 from a used stacked adhesive medical sensor come into contact with patient tissue. One or more new stacked adhesive layers 54A-C may be attached to the interior cover layer 62 during a remanufacturing process. One embodiment of such a remanufacturing process for producing the remanufactured stacked adhesive medical sensor 14 of FIG. 5 is also discussed below with reference to FIG. 8.

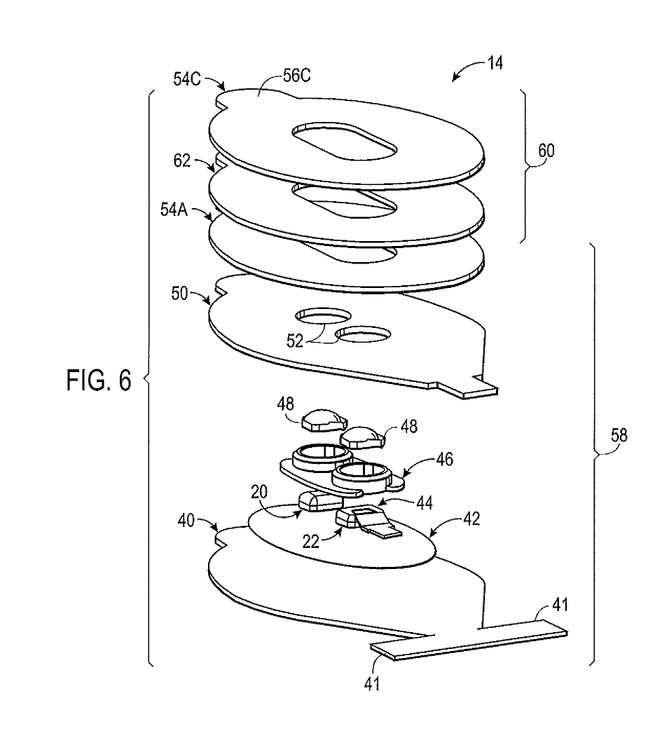

As will be discussed below, to obtain the remanufactured stacked adhesive medical sensors 14 shown in FIGS. 4 and 5, existing stacked adhesive layers from a used stacked adhesive medical sensor first may be removed, if any such stacked adhesive layers remain. In some embodiments, existing stacked adhesive medical sensors 54A-C may be left in place, but covered by an interior cover layer 62 to prevent a patient tissue site from contacting a layer from a used stacked adhesive medical sensor, which might have contacted another patient tissue. For example, as shown in FIG. 6, a remanufactured stacked adhesive medical sensor 14 may include existing components 58 that include all the components between and including the exterior foam layer 40 and one or more of the stacked adhesive layers 54A-C. Here, by way of example only, the existing stacked adhesive layer 54A remains. Certain new components 60 have been added during the remanufacturing process, including an interior cover layer 62 and at least one stacked adhesive layer 54C.

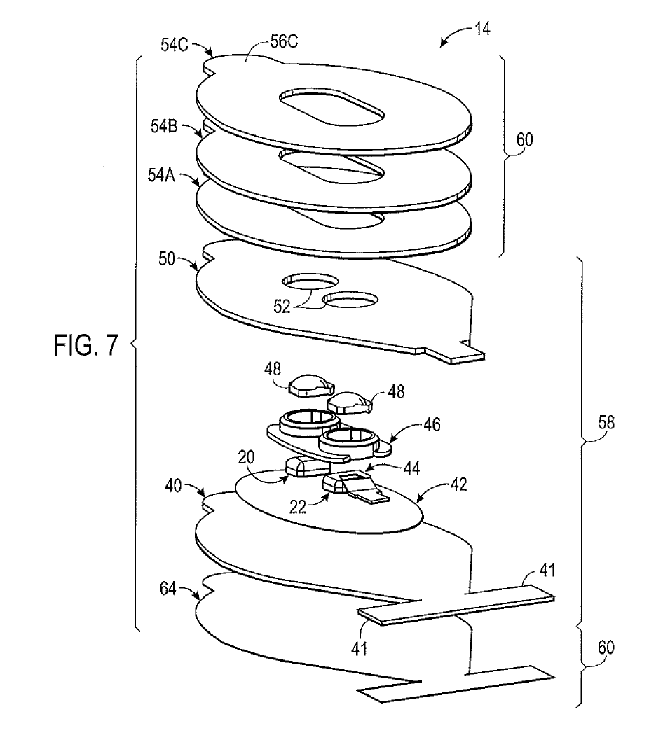

In another embodiment, represented by FIG. 7, existing components 58 from a used stacked adhesive medical sensor may include those between and including the exterior foam layer 40 and the interior foam layer 50. One or more stacked adhesive layers 54A-C may represent new components 60 added during the remanufacturing process. In addition, another new component 60 may be a non-patient-contacting-side exterior cover layer 64, which may attach to the exterior foam layer 40 to prevent the patient from contacting the existing 58 exterior foam layer 40.

The examples of stacked adhesive medical sensors 14 described above with reference to FIGS. 4-7 have been provided by way of example only, and should not be understood to be exclusive and/or exhaustive. Indeed, the various embodiments of FIGS. 4-7 may be combined. To provide just one example, other embodiments of the remanufactured stacked adhesive medical sensor 14 may include both an interior cover layer 62 (as shown in FIG. 5 or 6) and an exterior cover layer 64 (as shown in FIG. 7). Moreover, it should be understood that while not explicitly illustrated in FIGS. 4-7, the remanufactured stacked adhesive medical sensors 14 of FIGS. 4-7 may reuse the existing sensor cable 16 coupled to the emitter 20 and detector 22 of the used stacked adhesive medical sensors from which the remanufactured stacked adhesive medical sensors 14 are produced.

The remanufactured stacked adhesive medical sensors 14 described above with reference to FIGS. 4-7 may be remanufactured from a used stacked adhesive sensor according to a method shown in FIG. 8. Specifically, a flowchart 70 of FIG. 8 describing such a method may begin when a used stacked adhesive medical sensor is obtained (block 72). Such a used stacked adhesive medical sensor may have been used and/or discarded by a medical facility. In some embodiments, such a used stacked adhesive medical sensor may already have been sterilized, but in other embodiments, the used stacked adhesive medical sensor may be sterilized at the time that it is obtained in block 72.

The used stacked adhesive medical sensor next may be inspected preliminarily for apparent deficiencies that would make the sensor unsatisfactory for remanufacturing (block 74). If the used stacked adhesive medical sensor appears unsatisfactory (decision block 76), the sensor may be simply discarded (block 78). Otherwise, if the used stacked adhesive medical sensor does not preliminarily appear unsatisfactory (decision block 76), the number of times the used stacked adhesive medical sensor has been recycled may be determined (block 80).

The number of sensor recycle times may be determined in block 80 visually or by examining certain data stored in memory 27 in the connector 26 of the used stacked adhesive medical sensor. For example, in carrying out block 80, certain visual recycle indicators may be examined to ascertain a total recycle count for the used stacked adhesive medical sensor. For such embodiments, the number of times that the used stacked adhesive medical sensor has been used may appear in embossing 28, an ink indicator 30, on a cord tag 32, and/or as an exterior cover layer 64, and so forth. Additionally or alternatively, a recycle counter stored in the memory 27 of the connector 26 of the used stacked adhesive medical sensor may indicate the number of times the used stacked adhesive medical sensor has previously been recycled. If the used stacked adhesive medical sensor has already been recycled too many times (decision block 82), the used stacked adhesive medical sensor may be discarded (block 84). Otherwise, if the number of times the used stacked adhesive medical sensor has been recycled falls beneath a threshold (e.g., five times), the remanufacturing process of flowchart 70 may continue.

That is, in some embodiments, unused stacked adhesive layers 54A-C remaining on the used stacked adhesive medical sensor may be removed (block 86). Specifically, block 86 may take place to generate the remanufactured stacked adhesive medical sensors 14 illustrated in FIGS. 4, 5, and 7. Block 86 may be skipped to generate the remanufactured stacked adhesive medical sensor 14 illustrated in FIG. 6. The used stacked adhesive medical sensor next may be cleaned (block 88), by, for example, wiping the sensor with 70 percent isopropyl alcohol or any other suitable cleaning solution, or by other means such as pasteurization or application of ethylene oxide (EtO).

In some embodiments, the obtained used stacked adhesive medical sensor may store certain patient trend data in memory 27 located on the connector 26. If this trend data were to remain, the remanufactured stacked adhesive sensor 14 could wrongly indicate the subsequent patients' history. Accordingly, the trend data stored in the memory 27 on the connector 26 may be clear or overwritten and/or the trend feature may be disabled, for example, by setting a flag bit in a register of the memory 27 of the connector 26 (block 90).

In some embodiments, such as the embodiment discussed above with reference to FIGS. 5 and 6, a new interior cover layer 62 may be attached to the used interior foam layer 50 or, if the stacked adhesive layers 54 were not removed, over the outermost stacked adhesive layer 54 that remains on the used stacked adhesive medical sensor (block 92). Thereafter, new stacked adhesive layers (e.g., 54A-C), as well as a release liner to cover the outermost layer of adhesive, may be attached on the patient-contacting-side of the cover layer 62 or the existing components 58 of the used stacked adhesive medical sensor (block 94). It should be noted that, in some embodiments, an exterior cover layer 64 also may be attached to the exterior foam layer 40, in the manner shown in FIG. 7.

Having attached the new components 60 to the existing components 58 of the used stacked adhesive medical sensor, a series of sensor diagnostic tests may be performed (block 96). Such tests may include, for example, electrical tests (e.g., open-short, connectors, solder joint integrity, and so forth), detector 22 integrity tests, and/or tests on the wavelength of the emitter 20. A test for detector noise may indicate whether the Faraday shield 44 remains intact and at the proper placement. Additionally, in some embodiments, the emitter 20 wavelength may be tested to ensure that the wavelength of light emitted by the emitter 20 has not shifted beyond a point permitted by a calibration coefficient, which may be stored on the memory 27 of the connector 26. Although the used stacked adhesive medical sensor may not include a resistor indicating a bin to which the emitter 20 wavelength is assigned, data stored on the memory 27 of the connector 26 may be detected and, based on such data, the wavelength of the emitter 20 may be determined. From this determined wavelength, it may become apparent whether the wavelength of light currently being emitted by the emitter 20 has shifted beyond a point permitted by the calibration coefficient.

If any of the diagnostic tests of block 96 result in failure (decision block 98), the used stacked adhesive medical sensor may be sent to a further inspection procedure 100, such as described in greater detail below (block 100). Otherwise, if the used stacked adhesive medical sensor as past the sensor diagnostic tests of block 96 (decision block 98), the remanufacturing process may be completed and the remanufactured stacked adhesive medical sensor 14 may be prepared for use by a medical facility. Specifically, a recycle counter indicator of the remanufactured stacked adhesive medical sensor 14, such as the embossing 28, an ink indicator 30, a cord tag 32, and/or an exterior cover layer 64 may be incremented or memory 27 stored on the connector 26 may be incremented (block 102). Thereafter, the cord 16 may be wrapped, the remanufactured stacked adhesive medical sensor 14 may be placed into a package, and the package may be sterilized, pasteurized, or otherwise cleaned in any suitable manner (block 104). The remanufactured stacked adhesive medical sensor 14 then may be sent to a medical facility.

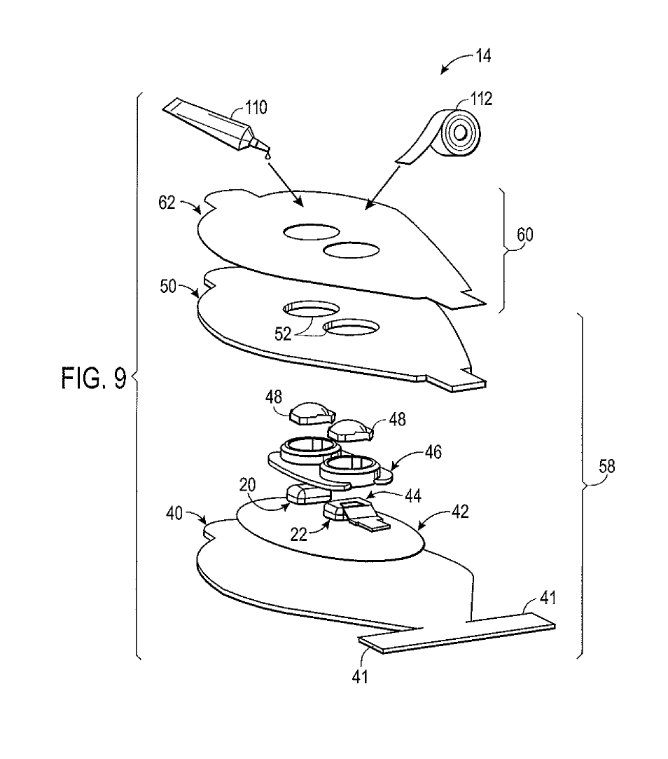

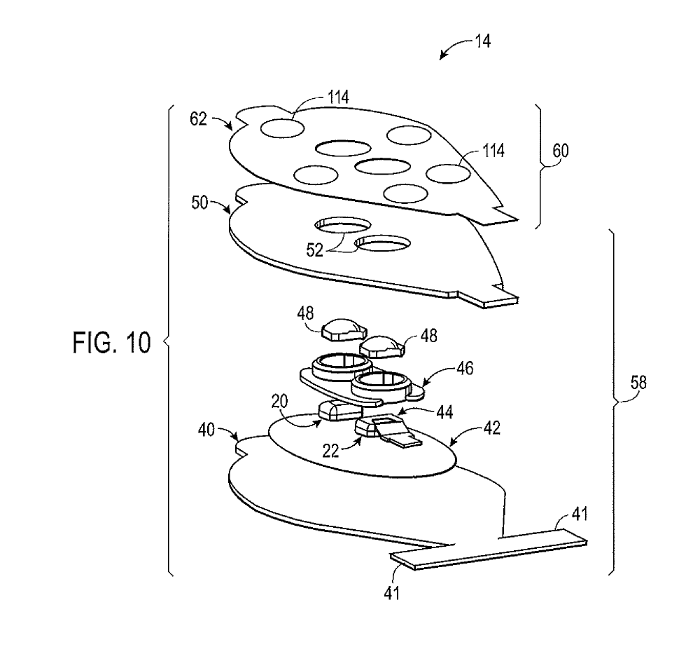

In some embodiments, the remanufactured stacked adhesive medical sensor 14 may not include any additional stacked adhesive layers 54A-C. Rather, as shown by FIGS. 9-11, some embodiments of the remanufactured stacked adhesive medical sensor 14 may use other techniques to attach to a patient tissue site. For example, as shown in FIG. 9, existing components 58 from a used stacked adhesive medical sensor may include the components between and including the exterior foam layer 40 and the interior foam layer 50. An interior cover layer 62 may represent a new component 60 that is attached to the interior foam layer 50. The cover layer 62 may not include any adhesive layer, so the remanufactured stacked adhesive medical sensor 14 may be packaged with certain adhesive-providing items such as adhesive gel 110 and/or bandage-type adhesives 112. In practice, the adhesive gel 110 or the bandage-type adhesives 112 may be used to attach the remanufactured stacked adhesive medical sensor 14 onto a patient tissue site. Alternatively, as shown in FIG. 10, a non-adhesive cover layer 62 may be a new component 60 that attaches to the interior foam layer 50. Certain adhesive dots 114 may be applied to the cover layer 62 to enable the cover layer 62 to attach to a patient tissue site.

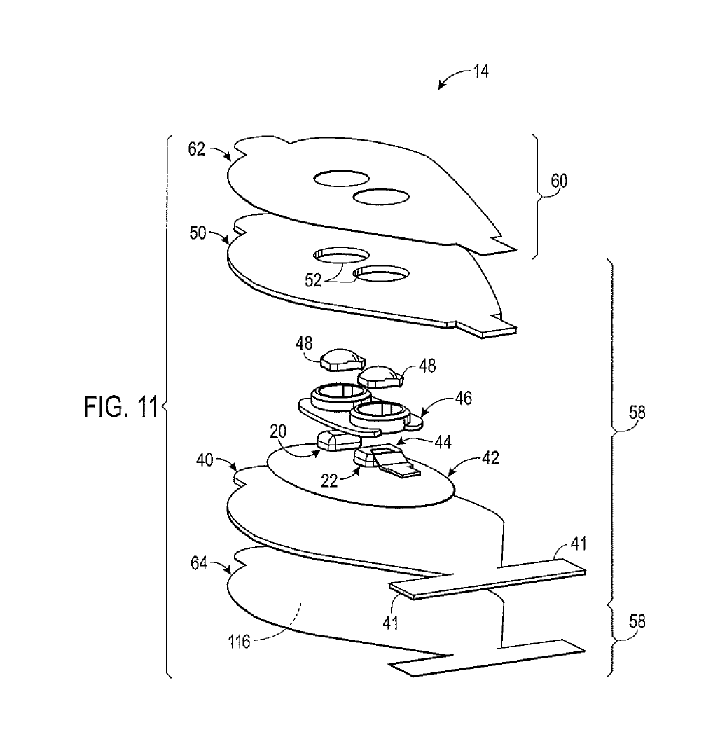

As shown in FIG. 11, some embodiments of the remanufactured stacked adhesive medical sensor 14 may attach to the patient tissue site at least partly by way of the elastic headband 36 shown in FIG. 3. That is, while the existing components 58 of the remanufactured stacked adhesive medical sensor 14 of FIG. 11 may include those components between and including the exterior foam layer 40 and the interior foam layer 50, an interior cover layer 62 without adhesive may be attached to the interior foam layer 50 as a new component 60. An exterior cover layer 64 may be attached to the exterior foam layer 40 to assist with attachment to the elastic headband 36 as a new component 60. The exterior cover layer 64 may have a headband-attaching exterior 116 for coupling to the elastic headband 36. For example, the exterior 116 may include an adhesive layer to attach to the elastic headband 36, may include a hook and loop material to hook into the elastic headband 36, and/or may be non-adhesive, but may have other adhesive materials attached (e.g., adhesive dots, bandage type adhesives, or adhesive gels).

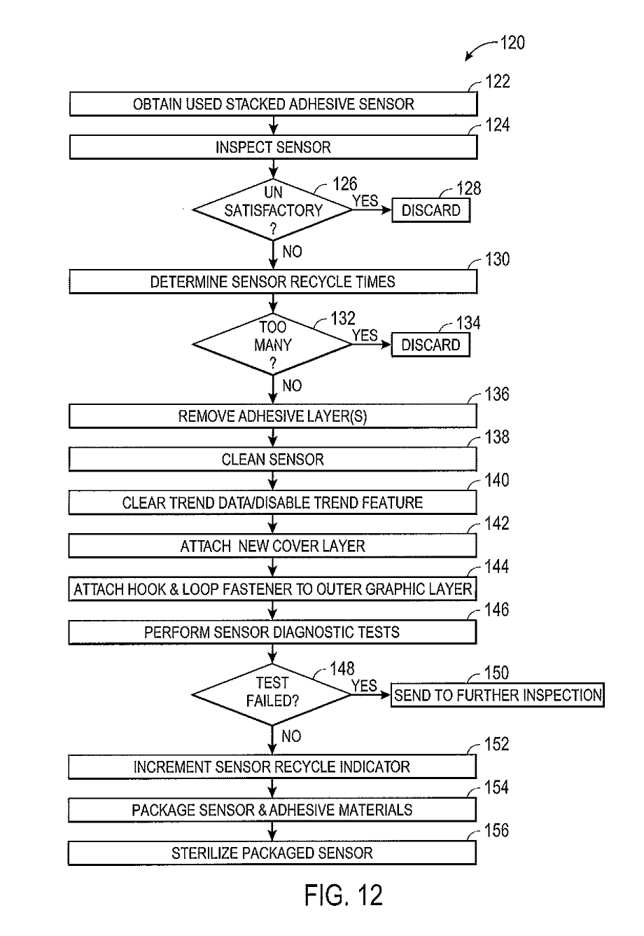

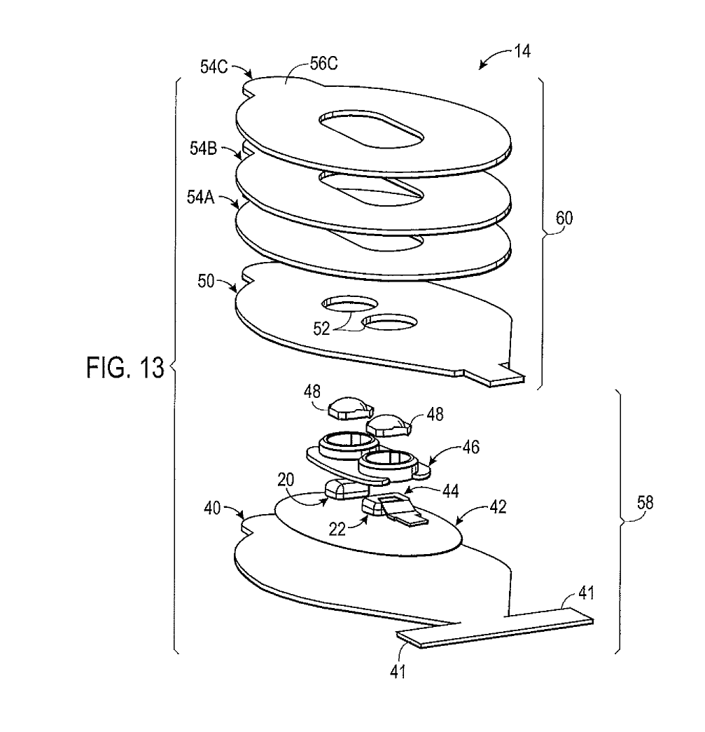

Remanufacturing a used stacked adhesive medical sensor to produce the remanufactured stacked adhesive medical sensors 14 shown in by FIGS. 9-11 may take place in a similar manner to the manufacturing process of FIG. 8. In particular, such a manufacturing process is illustrated by a flowchart 120 of FIG. 12. The flowchart 120 may begin in substantially the same manner as flowchart 70 of FIG. 8. Indeed, blocks 122-142 of the flowchart 120 may substantially take place in the same manner as blocks 72-92 of the flowchart 70, and thus the discussion of such blocks is not further addressed. In a subsequent step, the exterior cover layer 64, which may have a hook and loop fastener or other adhesive for joining to the elastic headband 36, may be attached to the exterior of the exterior foam layer 40 (block 144). In some embodiments, the actions of block 144 may be omitted.

Blocks 146-152 also may be performed in substantially the same manner as blocks 96-102 of FIG. 8. Thereafter, the remanufactured stacked adhesive medical sensor 14 may be packaged alongside separate adhesive materials, if needed (block 154). The packaged remanufactured stacked adhesive medical sensors next may be sterilized, pasteurized, or otherwise cleaned in any suitable manner (block 156).

In some embodiments, the remanufactured stacked adhesive medical sensor 14 may reuse existing components 58 between and including the exterior foam layer 40 through the windows 48, but not the interior foam layer 50. That is, the new components 60 of the remanufactured stacked adhesive medical sensor 14 of FIG. 13 may include a new interior foam layer 50 and one or more stacked adhesive layers 54A-C. In some embodiments, rather than the stacked adhesive layers 54A-C, the remanufactured stacked adhesive layer medical sensor 14 of FIG. 13 may include additional adhesives and/or an exterior cover layer 64 (not shown).

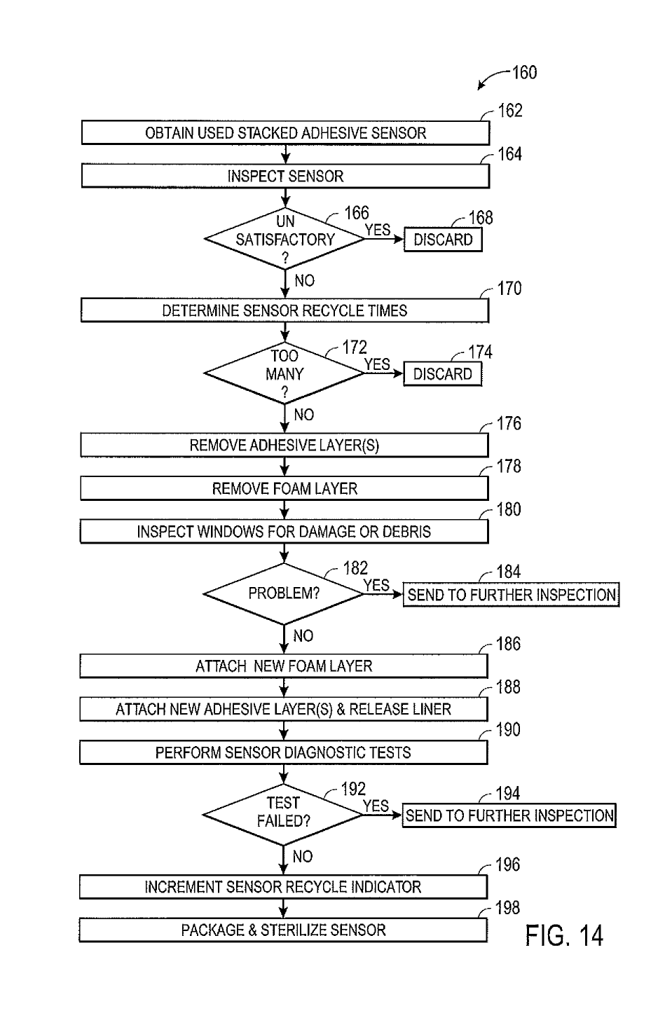

Because the interior foam layer 50 may be replaced in the remanufactured stacked adhesive medical sensor 14 shown in FIG. 13, the windows 48 in the semi-rigid optical mount 46 may be inspected. For example, FIG. 14 is a flowchart 160 describing an embodiment of a method for remanufacturing a used stacked adhesive medical sensor 14, such as the embodiment shown in FIG. 13. The flowchart 160 may begin in substantially the same way as the flowchart 70 of FIG. 8. That is, blocks 162-176 of the flowchart 160 of FIG. 14 may be substantially performed in the same manner as blocks 72-86 of FIG. 8. That is, after any remaining stacked adhesive layers 54A-C have been removed in block 176, the used interior foam layer 50 may be removed from the used stacked adhesive medical sensor.

Thereafter, the windows 48, semi-rigid optical mount 46, and mask layer 42 may be exposed. In this configuration, the windows 48 and/or the semi-rigid optical mount 46 may be inspected for damage or debris (block 180). If any damage or debris is detected (decision block 182), the used stacked adhesive medical sensor may be sent away for further inspection (block 184).

If no problems with the windows 48 or the semi-rigid optical mount 46 are detected (decision block 182), a new interior foam layer 50 may be attached to the exterior foam layer 40 over the emitter 20 and detector 22, such that the cutouts 52 surround the respective windows 48 (block 186). New stacked adhesive layers 54A-C in a release liner next may be attached to the new interior foam layer 50 (block 188). In some embodiments, in lieu of block 188, adhesive dots may be applied or other adhesives supplied in the package with the remanufactured stacked adhesive medical sensor 14. Blocks 190-196 may be performed in substantially the same manner as blocks 96-102 of FIG. 8. Thereafter, the remanufactured stacked adhesive medical sensor 14 may be packaged (alongside separate adhesive materials, if needed) and sterilized, pasteurized, or otherwise cleaned in any suitable manner (block 198).

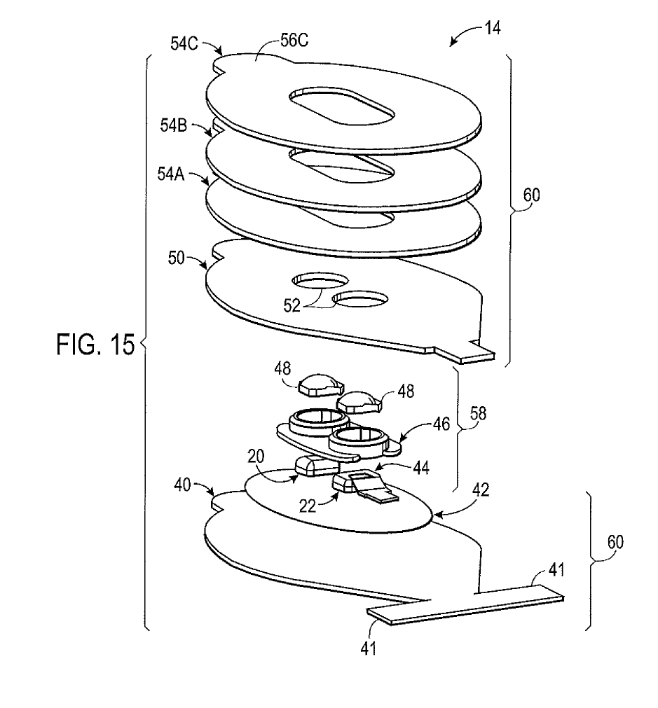

Another embodiment of the remanufactured stacked adhesive medical sensor 14 is represented by FIG. 15. In the embodiment of FIG. 15, the only existing components 58 remaining from a used stacked adhesive medical sensor may be the emitter 20, the detector 22, the Faraday shield 44, the semi-rigid optical mounts 46, and the windows 48. Indeed, the exterior foam layer 40, the mask layer 42, the interior foam layer 50, and one or more stacked adhesive layers 54A-C all may be new components 60 added or replaced during the remanufacturing process.

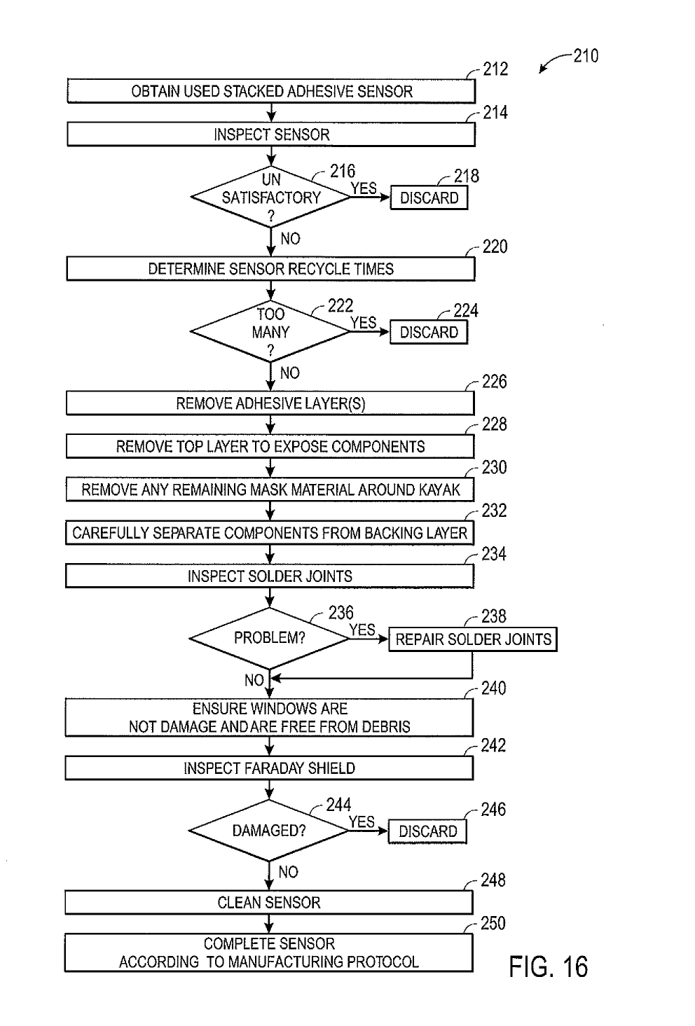

Such a remanufacturing process is described by a flowchart 210 of FIG. 16. The flowchart 210 may begin in substantially the same way as the flowchart 70 of FIG. 8. That is, blocks 212-226 of the flowchart 210 of FIG. 16 may be performed in substantially the same manner as blocks 72-86 of FIG. 8. Then, after any remaining stacked adhesive layers 54A-C have been removed in block 226, the interior foam layer 50 also may be removed (block 228), and any of the interior foam layer 50 that remains on or around the exposed components below may be removed, and any remaining mask layer 42 around on or around the semi-rigid optical mount 46 (the “kayak”) also may be removed (block 230).

The exposed components (the emitter 20, the detector 22, the Faraday shield 44, the semi-rigid optical mounts 46, and the windows 48) next may be carefully separated from the mask layer 42 and the exterior foam layer 40 (block 232). With all of the electrical components visible, the solder joints next may be inspected (block 234). If a problem is apparent (decision block 236), the solder joints may be repaired (block 238). Additionally, the windows 48 may be inspected for signs of damage or debris and, if such damage or debris is found, the used stacked adhesive medical sensor may be sent away for additional inspection (block 240).

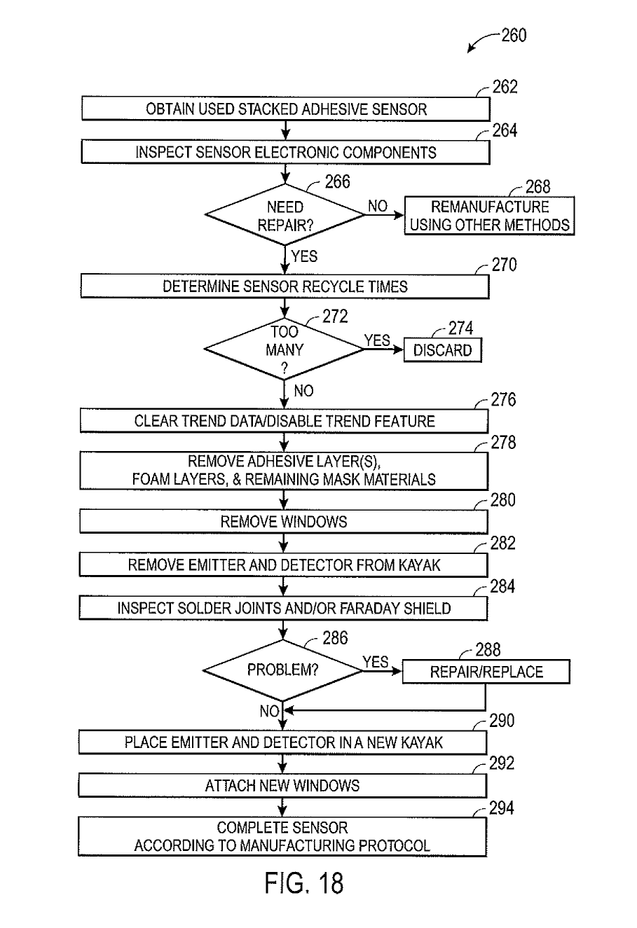

The Faraday shield 44 over the detector 22 next may be visually inspected through a window 48 over the detector 22 (block 242). If the Faraday shield appears damaged (decision block 244), the used stacked adhesive medical sensor may be discarded or may be sent away for further inspection (block 246) (e.g., for remanufacturing according to the method of FIG. 18). Thereafter, the used stacked adhesive medical sensor may be cleaned (block 248) according to any suitable technique, as discussed above. Now cleaned, these inspected existing components 58 of the used stacked adhesive medical sensor may form a basis around which the remanufactured stacked adhesive medical sensor 14 may be completed. In particular, the remanufactured stacked adhesive medical sensor 14 may be completed using any suitable manufacturing protocol (block 250), which may include the same standard manufacturing techniques used to manufacture an original stacked adhesive medical sensor, except that recycle indicators may be updated and/or trend data may be cleared or disabled.

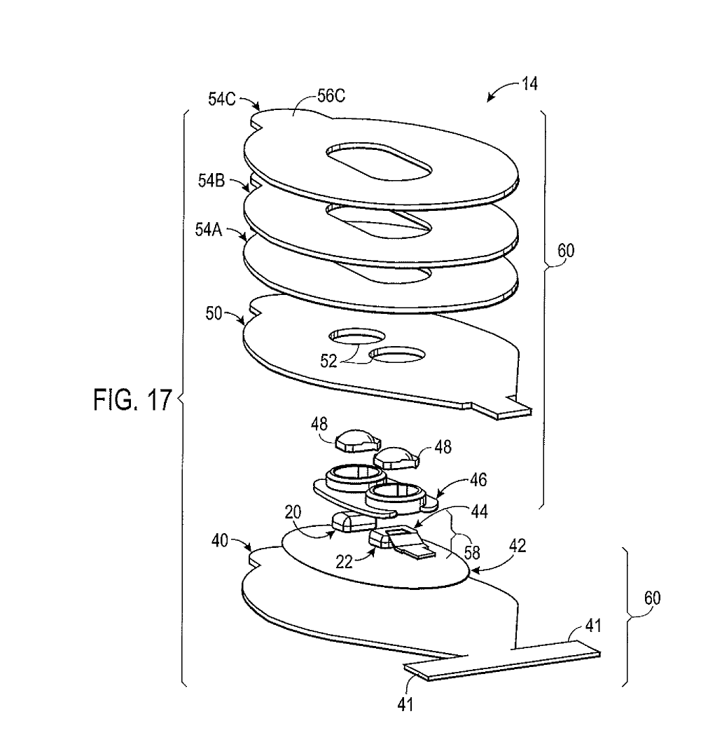

Finally, in some embodiments, substantially only the emitter 20 and detector 22 (as well as the cord 16 and connector 26) may be existing components 58 from a used stacked adhesive medical sensor that are reused in a remanufactured stacked adhesive medical sensor 14, as shown in FIG. 17. In particular, the remanufactured stacked adhesive medical sensor 14 of FIG. 17 recycles the emitter 20 and the detector 22, but replaces as new components 60 the exterior foam layer 40, the mask layer 42, the semi-rigid optical mount 46, the windows 48, the interior foam layer 50, and/or one or more stacked adhesive layers 54A-C or provides additional adhesive (e.g., in the manner of FIG. 9 or 10).

A flowchart 260 of FIG. 18 describes a method for producing the remanufactured stacked adhesive medical sensor 14 of FIG. 17. In particular, the flowchart 260 may begin when a used stacked adhesive medical sensor is obtained (block 262) and the electronic components are briefly inspected (block 264). If the electronic components of the used stacked adhesive medical sensor do not appear to be in need of repair (decision block 266), the used stacked adhesive medical sensor may be remanufactured using the other methods discussed herein (block 268).

The number of times the used stacked adhesive medical sensor has been recycled next may be determined (block 270) in substantially the same manner as block 80 of FIG. 8. If the used stacked adhesive medical sensor has already been recycled too many times (decision block 272), the used stacked adhesive medical sensor may be discarded (block 274). Otherwise, if the number of times the used stacked adhesive medical sensor has been recycled falls beneath a threshold (e.g., five times), the remanufacturing process of flowchart 260 may continue. The trend data stored in the memory 27 on the connector 26 may be cleared or overwritten and/or the trend feature may be disabled, for example, by setting a flag bit in a register of the memory 27 of the connector 26 (block 276).

Next, any unused stacked adhesive layers 54A-C remaining on the used stacked adhesive medical sensor, the exterior foam layer 40, the interior foam layer 50, and any remaining mask layer 42 material around the electronic components may be removed (block 278). With the electronic components of the used stacked adhesive medical sensor exposed, the windows 48 may be removed (block 280) before the emitter 20 and detector 22 are removed from the semi-rigid optical mount 46 (the “kayak”) (block 282). At this point or another point, these remaining components may be cleaned by, for example, wiping the sensor with 70 percent isopropyl alcohol or any other suitable cleaning solution, or by other means such as pasteurization or application of ethylene oxide (EtO).

With the emitter 20 and detector 22 exposed, the solder joints and/or the Faraday shield 44 next may be inspected (block 284). If a problem is apparent (decision block 286), the solder joints may be repaired and/or the Faraday shield 44 replaced (block 288). The emitter 20 and the detector 22 may be replaced into a new semi-rigid optical mount 46 (a new “kayak”) (block 290) and new windows 48 placed over the emitter 20 and detector 22 (block 292). Thereafter, the used stacked adhesive medical sensor may be cleaned according to any suitable technique, such as those mentioned above. These inspected existing components 58 of the used stacked adhesive medical sensor may now form a basis around which the remanufactured stacked adhesive medical sensor 14 may be completed. In particular, the remanufactured stacked adhesive medical sensor 14 may be completed using any suitable manufacturing protocol (block 294), which may include the same standard manufacturing techniques used to manufacture an original stacked adhesive medical sensor, except that recycle indicators may be updated and/or trend data may be cleared or disabled. In some embodiments, block 294 of FIG. 18 may take place in substantially the same manner as block 250 of FIG. 16.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

What is claimed is:

1. A remanufactured medical sensor comprising:
an exterior foam layer;
a mask layer coupled to the exterior foam layer;
an emitter coupled to the mask layer and configured to emit light into patient tissue;
a detector coupled to the mask layer and configured to detect light passing through the patient tissue;
a semi-rigid optical mount having recesses respectively configured to hold the emitter and the detector in place on the mask layer;
optical windows disposed over the recesses of the semi-rigid optical mount; and
an interior foam layer coupled to the exterior foam layer and disposed over the emitter and the detector, wherein the interior foam layer is configured to allow light to pass through the optical windows;
wherein the emitter and the detector are from a used stacked adhesive medical sensor and at least one of the exterior foam layer, the mask layer, the semi-rigid optical mount, the optical windows, or the interior foam layer is new.

2. The remanufactured medical sensor of claim 1, wherein the semi-rigid optical mount is from the used stacked adhesive medical sensor.

3. The remanufactured medical sensor of claim 2, wherein the exterior foam layer is from the used stacked adhesive medical sensor.

4. The remanufactured medical sensor of claim 3, wherein the optical windows and the interior foam layer are from the used stacked adhesive medical sensor.

5. The remanufactured medical sensor of claim 1, comprising a new adhesive layer configured to attach the remanufactured medical sensor to the patient tissue.

6. The remanufactured medical sensor of claim 1, comprising adhesive dots configured to attach the remanufactured medical sensor to the patient tissue.

7. The remanufactured medical sensor of claim 1, comprising an exterior cover layer coupled to the exterior foam layer, wherein the exterior cover layer is configured to prevent the patient from contacting the exterior foam layer.

8. The remanufactured medical sensor of claim 7, wherein the exterior cover layer is configured to attach to an elastic band configured to hold the remanufactured medical sensor against the patient tissue.

9. The remanufactured medical sensor of claim 1, comprising an interior cover layer coupled to the interior foam layer, wherein the interior cover layer is configured to prevent the patient from contacting the interior foam layer.

10. The remanufactured medical sensor of claim 1, comprising an adhesive layer coupled to the interior foam layer, wherein the adhesive layer and the interior foam layer are from the used stacked adhesive medical sensor, and comprising an interior cover layer coupled to the adhesive layer, wherein the interior cover layer is configured to prevent the patient from contacting the adhesive layer.

11. A method for remanufacturing a used stacked adhesive medical sensor comprising:
obtaining the used stacked adhesive medical sensor;
sterilizing the used stacked adhesive medical sensor;
performing at least one of clearing trend data stored on a memory device associated with the used stacked adhesive medical sensor and disabling a trend data feature of the memory device associated with the used stacked adhesive medical sensor;
attaching an adhesive layer onto the used stacked adhesive medical sensor;
performing a diagnostic test on the used stacked adhesive medical sensor to verify an operability of the used stacked adhesive medical sensor; and
providing an indication of a number of times the used stacked adhesive medical sensor has been remanufactured by providing or updating a recycle counter indicator associated with the used stacked adhesive medical sensor.

12. The method of claim 11, comprising determining a number of times the used stacked adhesive medical sensor has been remanufactured after the used stacked adhesive medical sensor is obtained and continuing the method only when the used stacked adhesive medical sensor has been remanufactured within a threshold number of times.

13. The method of claim 11, comprising removing an existing adhesive layer from the used stacked adhesive medical sensor before attaching the adhesive layer.

14. The method of claim 11, comprising attaching an interior cover layer onto the used stacked adhesive medical sensor before attaching the adhesive layer, wherein the adhesive layer is attached to the interior cover layer.

15. The method of claim 11, comprising replacing an existing interior foam layer of the used stacked adhesive medical sensor with a new interior foam layer.

16. The method of claim 11, comprising replacing an existing exterior foam layer of the used stacked adhesive medical sensor with a new exterior foam layer.

17. The method of claim 11, wherein the diagnostic test comprises an open-short test, a connector integrity test, a solder joint integrity test, a detector integrity test, or an emitter wavelength test, or any combination thereof.

18. The method of claim 11, wherein providing or updating the recycle counter indicator comprises embossing an indication of the number of times the used stacked adhesive medical sensor has been remanufactured, updating a recycle counter indicator located on the memory device associated with the used stacked adhesive medical sensor that indicates the number of times the used stacked adhesive medical sensor has been remanufactured, providing an ink indication of the number of times the used stacked adhesive medical sensor has been remanufactured, attaching a cord tag that indicates the number of times the used stacked adhesive medical sensor has been remanufactured, providing a cord clip that indicates the number of times the used stacked adhesive medical sensor has been remanufactured, or providing an exterior cover layer that indicates the number of times the used stacked adhesive medical sensor has been remanufactured, or any combination thereof.

19. The method of claim 11, comprising attaching an exterior cover layer onto the used stacked adhesive medical sensor, wherein the exterior cover layer is configured to prevent a patient from contacting a used exterior layer from the used stacked adhesive medical sensor.

20. A remanufactured stacked adhesive medical sensor assembly comprising:
a remanufactured stacked adhesive medical sensor comprising:
a plurality of components from a used stacked adhesive medical sensor, wherein the plurality of components includes an emitter configured to emit light into a patient tissue site and a detector configured to detect light from the patient tissue site; and
a new interior cover layer coupled to at least one of the plurality of components from the used stacked adhesive medical sensor, wherein the new interior cover layer is configured to be on or proximate to the patient; and
means for attaching the remanufactured stacked adhesive medical sensor to the patient.
